SEQUENCE LISTING (3 sequences)

SEQ ID NO: 1 - protein, 421 aa, Artificial Sequence
  Synthetic construct
  MISC_FEATURE: Tko EndoQ
MIVDADLHIHSRYSKAVSKAMTIPNLAENARFKGLEMVGTGDILNPNWEKELLKYTKKVDEGTYERNGIRFLLTTEVEDTRRVHHVLIFPNIETVREMRERLKPYSSDIESEGRPHLTLSAAEIADIANELDVLIGPAHAFTPWTSLYKEYDSLKEAYNGAKIHFLELGLSADSEMADMIKAHHKLTYLSNSDAHSPMPHRLGREFNRFEVNEATFEEIRKAILKRGRKIVLNAGLDPRLGKYHLTACSRCYTKYSLEEAKAFRWKCPKCGGRIKKGVRDRILELADTTERPKDRPPYLHLAPLAEIIAMVLGKGVETKAVRLVWERFLREFGSEIRVLVDVPVEELAKVHEEVAKAVWAYRKGKLIVISGGGGKYGEIKLPDEVRNARIEDLETIEVEVPNVEEKPKQRSITEFLRKSNK

SEQ ID NO: 2 - protein, 418 aa, Artificial Sequence
  Synthetic construct
  MISC_FEATURE: 9 degree N EndoQ
MLVDADLHLHSRYSKAVSKAMTIPNLAQNARFKGLGLVGTGDILNPHWEAELLRYAKKVDEGTYELNGIRFLLTTEVEDNRRVHHVLIFPSIETVREMREILKRYSTDIETEGRPHLSLSAAEIADIANDLDILIGPAHAFTPWTSLYKEYDSLKEAYRNARVHFLELGLSADSEMADMIKAHHRLTYLSNSDAHSPMPHRLGREFNRFEVEEVTFEEVRKAILRRGGRRIVLNAGLDPRLGKYHLTACSRCYAHYSLGEAKAFKWKCPKCGGRIKKGVKDRILELADTEERPKDRPPYLRLAPLAEIISMVIGKGIETKAVRLIWERFLRDFGSEIRVLVDVPVKELANVHEEVAKAIWAYRNGKLIVIPGGGGKYGEIKLPEEIRKARVEDLESVEVEIPEETEKPRQRSITDFLK

SEQ ID NO: 3 - protein, 263 aa, Artificial Sequence
  Synthetic construct
  MISC_FEATURE: Tk AGOG
MSLERFVKIKYQTNEEKADKLVEGLKELGIECARIIEEKVDLQFDALRHLRENLNDDETFIKLVIANSIVSYQLSGKGEDWWWEFSKYFSQNPPEKSIVEACSKFLPSSRTNRRLVAGKIKRLEKLEPFLNSLTLQELRRYYFENMMGLRNDIAEALGSPKTAKTVVFAVKMFGYAGRIAFGEFVPYPMEIDIPEDVRIKAYTERITNEPPVSFWRRVAEETGIPPLHIDSILWPVLGGKREVMERLKKVCEKWELVLELGSL

(19) United States
(12) Patent Application Publication
(10) Pub. No.: US 2022/0282233 A1
(43) Pub. Date: Sep. 8, 2022

(54) Cleavage of Single Stranded DNA Having a Modified Nucleotide

(71) Applicant: New England Biolabs, Inc., Ipswich, MA (US)

(72) Inventors: Andrew F. Gardner, Manchester, MA (US); Kelly M. Zatopek, Wilmington, MA (US)

(73) Assignee: New England Biolabs, Inc., Ipswich, MA (US)

(21) Appl. No.: 17/637,430

(22) PCT Filed: Aug. 21, 2020
(86) PCT No.: PCT/US2020/047504

§ 371 Date: Feb. 22, 2022

Related U.S. Application Data
(60) Provisional application No. 62/890,291, filed on Aug. 22, 2019.

(51) Int. Cl. C12N 9/22; C12N 15/10; C12Q 1/6806; C12N 9/24; C07K 1/10

(52) U.S. Cl. CPC C12N 9/22; C12N 15/1065; C12N 15/1068; C12Q 1/6806; C12Y 301/21; C12N 9/2497; C12Y 302/02; C07K 1/10; C12Q 1/6853

(57) ABSTRACT

Methods are provided that, for example, include (a) combining ssDNA containing a modified nucleotide (e.g., a ssDNA with a modified nucleotide proximate to its 5′ end) with a DNA cleavage enzyme capable of cleaving the ssDNA at the modified nucleotide (e.g., to generate a first ssDNA fragment having a 3′OH and a second ssDNA fragment having the modified nucleotide); wherein the ratio of enzyme to DNA substrate is less than 1:1 molar ratio (m/m); and (b) cleaving at least 95% of the ssDNA at the modified nucleotide. In some embodiments, a method may comprise (a) combining (i) a ssDNA comprising a modified nucleotide (e.g., proximate to its 5′ end) with (ii) a DNA cleavage enzyme capable of cleaving the ssDNA at the modified nucleotide (e.g., to generate (after cleavage) a first ssDNA fragment having a 3′OH and a second ssDNA fragment comprising the modified nucleotide) wherein the ratio of enzyme to DNA substrate is less than 1:1 molar ratio and cleaving at least 95% of the ssDNA at the modified nucleotide. In some embodiments, methods provided herein may include (a) combining (i) a ssDNA (1) immobilized on a substrate and (2) comprising a modified nucleotide with (ii) a ssDNA cleaving enzyme capable of cleaving the ssDNA at the modified nucleotide (e.g., to generate (after cleavage) a first ssDNA fragment having a 3′OH and a second ssDNA fragment comprising the modified nucleotide) ; and (b) cleaving the immobilized ssDNA to release the second single stranded DNA fragment from the substrate. At least 95% (m/m) of an ssDNA comprising a modified nucleotide may be cleaved in less than 60 minutes.

### BACKGROUND

Traditional phosphoramidite chemistry synthesizes DNA from the 3′-5′ direction on a solid support microarray. Release of oligonucleotides is typically by chemical cleavage such as such as 35% NH₄OH treatment for 2 hours (Kosuri, et al., Nat Methods, 11, 499-507 (2014); Cleary, et al., Nat Methods, 1, 241-248 (2004); Tian, et al., Nature, 432, 1050-1054 (2004)).

More recently enzymatic methods have been used to synthesize long oligonucleotides using modified terminal deoxynucleotidyl transferase (TdT) and modified nucleotide terminators. In this method TdT builds an oligonucleotide from an immobilized primer in the 5′-3′ direction by incorporating a specific nucleotide terminator base on the 3′ end of a tethered oligonucleotide. After washing and deprotection of the nucleotide terminator blocking group, the next nucleotide terminator is added. Cycles of incorporation by TdT, washing and deprotection synthesizes oligonucleotides on a solid support. However, these methods must efficiently remove the synthesized oligonucleotides from the solid support. Currently methods use photoactivation to release oligonucleotides from a solid support. Improved methods to release oligonucleotides from solid supports are needed to maximize yield and efficiency.

Although the number of oligonucleotides that can be produced in a pool by oligonucleotide arrays is large, their individual concentrations are very low and require an additional amplification step. PCR amplification directly on the oligonucleotide array can amplify oligonucleotides, however, efficiency may be lower than in solution PCR (Kosuri, et al. (2014) Nat Methods, 11, 499-507.). Therefore, releasing the oligonucleotides from the array could improve subsequent PCR amplification of the library.

Existing enzyme methods for releasing immobilized DNA generally have a significant preference for double stranded DNA (dsDNA) (such as, EndoV, RNase H2 and glycosylase/lyases). Moreover, it has been reported for some enzyme systems that enzyme concentrations required for cleavage significantly exceeded the single stranded (ss) oligonucleotide concentration which suggested that the enzymes would be impractical for routine use (see for example Shiraishi, et al., Nucleic Acids Res, 43, 2853-2863 (2015)). In some cleavage protocols e.g. chemical cleavage, cleavage of single stranded DNA (ssDNA) from a solid support is inefficient (for example having reaction times of 10 hours or more).

### SUMMARY

Methods are provided that, for example, include (a) combining ssDNA containing a modified nucleotide (e.g., a ssDNA with a modified nucleotide proximate to its 5′ end) with a DNA cleavage enzyme capable of cleaving the ssDNA at the modified nucleotide (e.g., to generate a first ssDNA fragment having a 3′OH and a second ssDNA fragment having the modified nucleotide); wherein the ratio of enzyme to DNA substrate is less than 1:1 molar ratio (m/m); and (b) cleaving at least 95% of the ssDNA at the modified nucleotide. In some embodiments, a method may comprise (a) combining (i) a ssDNA comprising a modified nucleotide (e.g., proximate to its 5′ end) with (ii) a DNA cleavage enzyme capable of cleaving the ssDNA at the modified nucleotide (e.g., to generate (after cleavage) a first ssDNA fragment having a 3′OH and a second ssDNA fragment comprising the modified nucleotide) wherein the ratio of enzyme to DNA substrate is less than 1:1 molar ratio and cleaving at least 95% of the ssDNA at the modified nucleotide. In some embodiments, methods provided herein may include (a) combining (i) a ssDNA (1) immobilized on a substrate and (2) comprising a modified nucleotide with (ii) a ssDNA cleaving enzyme capable of cleaving the ssDNA at the modified nucleotide (e.g., to generate (after cleavage) a first ssDNA fragment having a 3′OH and a second ssDNA fragment comprising the modified nucleotide) ; and (b) cleaving the immobilized ssDNA to release the second single stranded DNA fragment from the substrate. At least 95% (m/m) of an ssDNA comprising a modified nucleotide may be cleaved in less than 60 minutes.

A method, in some embodiments, may include one or more of the following:
(a) cleaving at least 95% of the ssDNA in less than 60 minutes;
(b) the ssDNA comprising a modified nucleotide further comprises the modified nucleotide proximate to the 5′ end of the ssDNA;
(c) the ssDNA cleaving enzyme comprises an endonuclease, the ssDNA is attached (e.g., immobilized) to a solid substrate (e.g., at the 5′ end of the ssDNA, and/or the modified nucleotide is proximate to the the 5′ end (e.g., the bound 5′ end)), and cleaving further comprises releasing from the substrate a fragment of the ssDNA comprising the modified nucleotide and nucleotides 3′ to the modified nucleotide;
(d) prior to step (a) generating the ssDNA by reverse transcribing an RNA;
(e) the ssDNA contains a modified nucleotide proximate to its 5′ end further comprises a label at a 3′ end where for example, the label is a fluorescent tag;
(f) the ssDNA contains a modified nucleotide proximate to its 5′ end and the 5′ end is immobilized on a solid support;
(g) the solid support is a bead;
(h) the solid support is plastic plate with (e.g., comprising) wells;
(i) the solid support is a two-dimensional surface on which the ssDNA forms an array;
(j) the ssDNA cleaving enzyme comprises a thermophilic endonuclease for example, an archaeal endonuclease with a preference for cleaving ssDNA, for example EndoQ or AGOG;
(k) the ssDNA cleaving enzyme comprises a fusion protein where for example, an endonuclease is fused to SNAP-tag which may in turn be bound to the solid substrate;
(l) the modified nucleotide is an 8-oxoguanine (8oxoG) or deoxyuridinel (dU) or deoxyinosine (dI) or deoxyxanthosine (dX) or tetrahydrofuran (THF) site;
(m) the single stranded oligonucleotide is a product of ssDNA synthesis and optionally contains a barcode of randomly generated nucleotides;
(n) the single stranded DNA is or comprises an aptamer;
(o) the single strand synthesis is chemical or enzymatic.

Compositions are provided that include an artificial mixture of a ssDNA-cleaving archaeal endonuclease or glycosylase and a synthetic DNA substrate comprising a modified nucleotide. A composition may have one or more of the following:
(a) a synthetic DNA substrate immobilized on a solid substrate;
(b) the solid substrate is selected from a bead, a well in a multi-well dish and a 2-dimensional array surface;
(c) the modified nucleotide is selected from the group consisting of THF site, dU, dI, 8-oxoG and dX; and
(d) the endonuclease is a fusion protein that may comprise a SNAP-tag.

### BRIEF DESCRIPTION OF THE FIGURES

FIG. 1 shows a workflow to release modified synthesized ssDNA oligonucleotides from a solid support with an endonuclease having ssDNA>dsDNA activity. As illustrated, a method for use in DNA synthesis that includes (1) attaching to a solid support one end (e.g., the 5′ end, marked “0”) of a ssDNA comprising at or near its 3′ end a modified base (“X”) (e.g., dI, dU, 8-oxo-dG, dX, THFP), (2) extending the ssDNA synthetically from its free end to form an extension oligonucleotide (e.g. using TdT or chemical extension (see for example Perkel, (2019) Nature 566, 565)), (3a) contacting the extended ssDNA with an endonuclease with ssDNA cleaving activity (3b) to cleave the extended ssDNA at a position adjacent to the modified base forming a free cleavage product comprising the modified base and the extension oligonucleotide and a bound oligo fragment that remains tethered to the support, and (4) eluting the cleavage product from the solid support.

FIG. 2 shows a workflow to capture and enrich nucleic acids with modified ssDNA oligonucleotides and an endonuclease with ssDNA>dsDNA cleavage activity. As illustrated, release of the immobilized modified ssDNA oligonucleotides is achieved using an endonuclease with a preference for ssDNA cleavage activity. Cleaved oligos can be used for gene assembly methods, next generation sequencing (e.g., Illumina, PacBio, Oxford Nanopore), PCR primers or other techniques. Solid supports may be selected from or comprise beads, plates, and/or materials. Coupling a capture oligonucleotide to a solid support may be achieved using techniques such as streptavidin:biotin binding, SNAP-tag, CLIP-tag, click chemistry among others. A capture bead may comprise a complementary capture sequence, which may be or comprise, for example, poly(dT), poly(A), mRNA, a custom sequence specific to the intended target DNA or RNA species, and/or a library of sequences to enrich for certain DNA or RNA species (e.g., exons).

FIG. 3A-3D shows that Thermococcus sp 9° N (9° N) EndoQ and Thermococcus kodakarensis (Tko) EndoQ both show a preference for cleaving ssDNA containing a dU.

FIG. 3A shows an experimental design for measuring 9° N EndoQ cleavage activity of DNA containing a dU.

FIG. 3B shows that 9° N EndoQ ssDNA-dU is substantially greater and more rapid than dsDNA-dU cleavage. Cleavage of ssDNA was substantially complete by 2 minutes after initiation of the reaction whereas even after 10 minutes, dsDNA was not completely cleaved. The fraction of ssDNA-dU (open circles) and dsDNA-dU (closed circles) cleaved product was calculated, plotted and fit to an exponential rise equation (y=m1+m2*(1−exp(−m3*x)). The rate of ssDNA-dU cleavage was 5.7 min⁻¹ and dsDNA-dU was 0.16 min⁻¹. The ratio of ssDNA-dU:dsDNA-dU activity by 9° N EndoQ was 35.

FIG. 3C shows an experimental design for measuring Tko EndoQ cleavage activity of DNA comprising dU.

FIG. 3D shows that Tko EndoQ ssDNA-dU is substantially greater and more rapid than dsDNA-dU cleavage. The fraction of ssDNA-dU (open circles) and dsDNA-dU (closed circles) cleaved product was calculated, plotted and fit to an exponential rise equation (y=m1+m2*(1−exp(−m3*x)). The rate of ssDNA-dU cleavage was 0.3 min⁻¹ and dsDNA-dU was 0.03 min⁻¹. The ratio of ssDNA-dU:dsDNA-dU activity by Tko EndoQ was 10.

FIG. 4A-4D shows that 9° N EndoQ and TKO EndoQ both show a preference for cleaving ssDNA comprising dI

FIG. 4A shows an experimental design for measuring 9° N EndoQ cleavage activity of DNA comprising idI.

FIG. 4B shows that 9° N EndoQ ssDNA-dI is substantially greater and more rapid than dsDNA-dI cleavage. The fraction of ssDNA-dI (open circles) and dsDNA-dI (closed circles) cleaved product was calculated, plotted and fit to an exponential rise equation (y=m1+m2*(1−exp(−m3*x)). The rate of ssDNA-dI cleavage was 1.0 min⁻¹ and dsDNA-dI was 0.2 min⁻¹. The ratio of ssDNA-dI:dsDNA-dI activity by 9° N EndoQ was 5.

FIG. 4C shows an experimental design for measuring Tko EndoQ cleavage activity of DNA comprising dI.

FIG. 4D shows that Tko EndoQ ssDNAd-l is substantially greater and more rapid than dsDNA-dI cleavage. The fraction of ssDNA-dI (open circles) and dsDNA-dI (closed circles) cleaved product was calculated, plotted and fit to an exponential rise equation (y=m1+m2*(1−exp(−m3*x)). The rate of ssDNA-dI cleavage was 0.45 min⁻¹ and dsDNA-dI was 0.013 min⁻¹. The ratio of ssDNA-dI:dsDNA-dI activity by Tko EndoQ was 35.

FIG. 5A-5B shows that AGOG shows a preference for cleaving ssDNA comprising 8-oxoG.

FIG. 5A shows an experimental design for determining AGOG cleavage activity of DNA substrate.

FIG. 5B shows that AGOG cleaved ssDNA-8oxoG with 3.5-fold greater activity than cleavage of dsDNA-8oxoG cleavage activity.

The fraction of ssDNA-8oxoG (open circles) and dsDNA-8oxoG (closed circles) cleaved product was calculated, plotted and fit to an exponential rise equation (y=m1+m2*(1−exp(−m3*x)). The rate of ssDNA-8oxoG cleavage was 4.3 min⁻¹ and dsDNA-8oxoG was 1.2 min⁻¹.

FIG. 6A-6C shows that RNase H2 cleavage activity of dDNAs is more rapid (completed with less than a second) than cleavage of ssDNA substrate (completed within about 2 hours). This contrasts with the results in FIG. 3A-5B, which show ssDNA cleavage outpacing dsDNA cleavage.

FIG. 6A shows an experimental design for determining RNaseH2 cleavage activity of DNA substrate

FIG. 6B-6C shows the fraction of (B) dsDNA-rG (closed circles) and (C) ssDNA-rG (open circles) at various incubation times. The amount of cleaved product was calculated, plotted and fit to an exponential rise equation (y=m1+m2*(1−exp(−m3*x)). The rate of ssDNA-rG cleavage was 0.03 min⁻¹ and dsDNA-rG was 3,500 min⁻¹. The ratio of ssDNA-rG:dsDNA-rG activity by 9° N RNaseH2 was 8.5 x 10⁻⁶.

FIG. 7A-7E shows that 9° N EndoQ and Tko EndoQ are similarly effective at cleaving ssDNA with a modified dU or dI from magnetic beads.

FIG. 7A shows an experimental design for determining EndoQ cleavage activity of DNA substrate containing a dU modification from beads.

FIG. 7B shows how the efficiency of cleavage of ssDNA-dU by 9° N EndoQ varies with concentration of the enzyme.

FIG. 7C shows ssDNA-dU cleavage from magnetic beads by 9N Endo Q (filled circles) or Tko EndoQ (filled squares). “No enzyme” control (open circles).

FIG. 7D shows an experimental design for determining EndoQ cleavage activity of DNA substrate containing a dI modification from beads.

FIG. 7E shows ssDNA-dI cleavage from magnetic beads by 9N Endo Q (filled circles). “No enzyme” control (open circles).

FIG. 8A-8D shows that 2 different EndoQs can effectively cleave DNA substrate containing two different modified nucleotides from multiwell plates.

FIG. 8A shows an experimental design for determining EndoQ cleavage activity of ssDNA-dU DNA substrate from a plate surface.

FIG. 8B shows Cleavage of ssDNA-dU from a plate by 9° N and Tko EndoQ. (A) ssDNA-dU cleavage from a plate by 9° N Endo Q (filled circles), Tko EndoQ (open circles) or “no enzyme control” (open squares) over time

FIG. 8C shows an experimental design for determining EndoQ cleavage activity of ssDNA-dI DNA substrate from a plate surface.

FIG. 8D. shows Cleavage of ssDNA-dI from a plate by 9° N and Tko EndoQ. (A) ssDNA-dI cleavage from a plate by 9° N Endo Q (filled circles), Tko EndoQ (open circles) or no enzyme control (open squares) over time.

FIG. 9A-9B shows that at least 90% of the immobilized ssDNA-dU was cleaved using less than or equal 1:1 molar ratio of EndoQ:immobilized ssDNA-dU using a ssDNA-dU-3′-FAM substrate and 9° N EndoQ.

FIG. 9A shows how 30 nM 9° N EndoQ results in substantially 100% cleavage of ssDNA.

FIG. 9B shows the molar ratio of 9° N EndoQ to immobilized ssDNA-dU.

### DETAILED DESCRIPTION

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative). All publications, patents, and patent applications mentioned in this specification are herein incorporated by reference to the same extent as if each individual publication, patent, or patent application was specifically and individually indicated to be incorporated by reference.

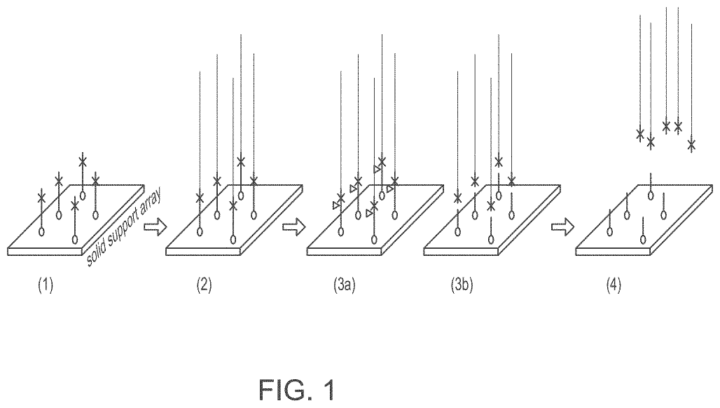

Solutions are provided to the problem of cleaving ssDNA at a targeted site where the cleaved portion or fragment released after cleavage retains a terminal modified nucleotide at the 5′ cleaved end. As illustrated in FIG. 1, a method, in some embodiments, may include attaching to a solid support a ssDNA comprising, in a 5′ to 3′ direction, a 5′ end, a modified nucleotide (“X”), and a 3′ end. A ssDNA, in some embodiments, may include at the 5′ end a binding moiety capable of binding a solid support, for example, streptavidin, biotin, SNAP-tag, CLIP-tag, and/or benzyl-G. FIG. 1 shows (1) attaching a ssDNA (●—X-3′) to a solid support, (2) extending the ssDNA from the 3′ end (gray lines), (3a) contacting the ssDNA with a ssDNA cleaving enzyme (e.g., an endonuclease) (3b) to form a ssDNA fragment that remains bound to the solid support and release a ssDNA fragment comprising the modified nucleotide, and eluting the released ssDNA fragment. This may be performed in an array format and the eluted ssDNA fragments may be used for any desired application. For example, eluted ssDNA fragments may be used for oligonucleotides for gene synthesis. In the context of the present disclosure, “modified nucleotides” refers to any noncanonical nucleoside, nucleotide or corresponding phosphorylated versions thereof. Modified nucleotides may include one or more backbone or base modifications. Examples of modified nucleotides include dI, dU, 8-oxo-dG, dX, and THF. Additional examples of modified nucleotides include the modified nucleotides disclosed in U.S. Patent Publication Nos. US20170056528A1, US20160038612A1, US2015/0167017A1, and US20200040026A1. Modified nucleotides may include naturally or non-naturally occurring nucleotides.

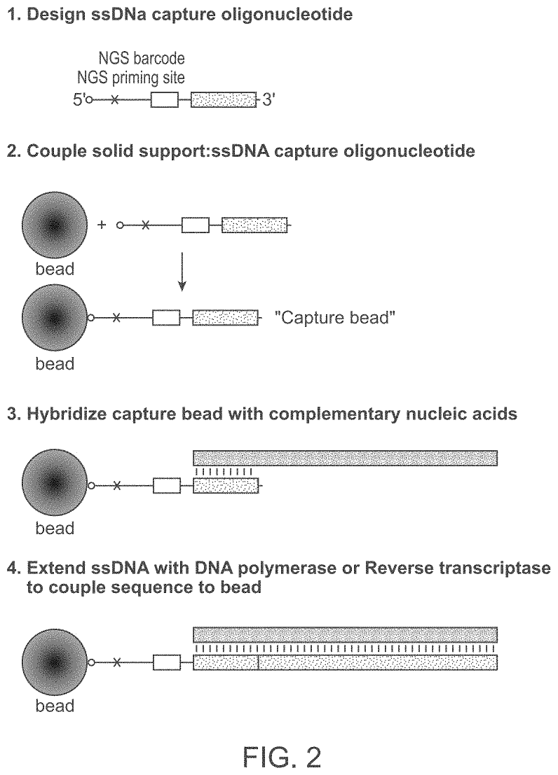

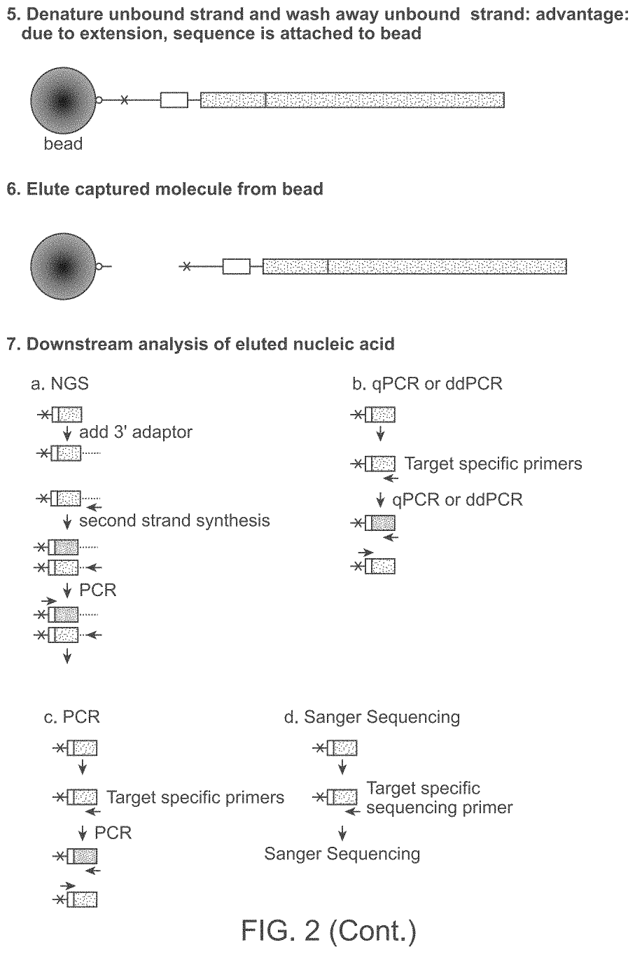

In some embodiments, a ssDNA may comprise, in a 5′ to 3′, a 5′ end, a modified nucleotide (“X”), a barcode or priming site (e.g., a next generation sequencing (NGS) barcode or NGS priming site), a complementary capture sequence, and a 3′ end (FIG. 2, step 1). A 5′ end may comprise a substrate binding moiety (e.g., biotin or benzyl guanidine). A capture bead may comprise a capture sequence, which may be or comprise, for example, poly(dT), poly(A), mRNA, a custom sequence specific to the intended target DNA or RNA species, and/or a library of sequences to enrich for certain DNA or RNA species (e.g., exons). A ssDNA may be coupled to a bead (FIG. 2, step 2), plate (e.g., well of a plate comprising multiple wells), or other materials (e.g., macro structures and/or insoluble materials) to form a capture bead. A capture bead may be used as bait to attract a polynucleotide complementary or generally complementary to the capture sequence. The single strand bait supports hybridization of the bound sequences with one or more complementary nucleic acids comprising or potentially comprising a complementary sequence (FIG. 2, step 3). As shown in FIG. 2 (step 4), once hybridized, the bound ssDNA sequence may be extended in a template-dependent manner (e.g., using a DNA polymerase or reverse transcriptase) to produce an extension product comprising, in a 5′ to 3′ direction, a bound 5′ end, a modified nucleotide, a barcode and/or priming site, a complementary capture sequence, a sequence complementary to the captured nucleic acid, and a 3′ end. A captured complementary nucleic acid may comprise, in some embodiments, one or more modified nucleotides (e.g., to facilitate removal of the captured complementary nucleic acid during step 6 (below)). In some embodiments, a complementary nucleic acid may be separated from the extension product (e.g., by thermal or chemical denaturation). An advantage of methods according to some embodiments is the nascent portion of the extension product is attached to the support (e.g., bead), permitting optional washing and other manipulation (FIG. 2, step 5). When desired, the nascent portion of the extension product may be released from the support. For example, the extension product may be contacted with a ssDNA cleaving enzyme (an endonuclease) that cleaves at or proximal to a modified nucleotide to form (a) a ssDNA fragment that remains bound to the support (e.g., bead) comprising, for example, the 5′ end of the original ssDNA, and (b) an unbound ssDNA fragment that is released. The unbound ssDNA fragment may comprise the modified nucleotide, the barcode or priming site, the capture sequence, the extenions sequence (i.e., complementary to the (formerly) captured complementary nucleic acid), and the 3′ end (FIG. 2, step 6). The unbound ssDNA fragment (comprising the sequence complementary to the captured molecule) may be eluted from the bead, for example, with a wash buffer (FIG. 2, step 6). An unbound ssDNA fragment may be analyzed by next generation sequencing (e.g., Illumina, PacBio, Oxford Nanopore) (FIG. 2, step 7a). As illustrated, the unbound ssDNA may be combined with a 3′ adapter (e.g., by ligation, TdT, or poly(A) polymerase), followed by second strand synthesis and PCR amplification. In some embodiments, an unbound ssDNA fragment may be analyzed by quantitative PCR (qPCR) or DROPLET DIGITAL™ PCR (ddPCR™) (FIG. 2, step 7b) or conventional PCR (FIG. 2, step 7c) with, for example, target specific primers (e.g., p53 oncogene primers). An unbound ssDNA may be analyzed, in some embodiments, by Sanger sequencing (e.g., for mutation detection) with target specific primers (e.g., p53 oncogene primers) (FIG. 2, step 7d).

Benefits of achieving cleavage in this manner is that immobilized ssDNA can be released from a solid surface while retaining a tag for further manipulation. Another benefit of embodiments of the methods described herein is that the ratio of enzyme to substrate is less than 1:1. Another benefit of embodiments of the methods described herein is that ssDNA is cleaved with a significant preference over dsDNA that is a useful feature in sequencing protocols. Another benefit of embodiments of the methods described herein that the cleavage reaction requires only a single enzyme.

Another benefit of embodiments of the methods described herein is the presence of a 3′OH on the cleaved end of the ssDNA cleavage product that no longer includes the modified nucleotide. Embodiments of the methods enable more efficient cleavage of modified ssDNA from a solid support for oligonucleotide synthesis, gene assembly and nucleic acid capture and enrichment.

Embodiments of the methods of cleavage of modified ssDNA, where for example, the DNA is immobilized on a solid support include; cleavage of captured and extended ssDNA/RNA from beads; cleavage of captured and extended ssDNA/RNA from beads from single cells; cleavage of chemically synthesized oligonucleotides from solid support array; cleavage of enzymatically synthesized oligonucleotides from solid support array; cleavage of barcoded oligonucleotides from a solid support; cleavage of ssDNA: protein from a solid support; and/or cleavage of an aptamer pool from a solid support.

Examples of ssDNA cleaving enzymes with a preference for ssDNA over dsDNA, that preferably have a reaction time of less than 10 hours and preferably an effectiveness at a molar ratio of enzyme to substrate that is less than 1:1 include the following: EndoQ, for example, thermostable EndoQs such as 9° N EndoQ, Tko Endo Q; 8-Oxoguanine DNA Glycosylase (AGOG), Argonautes (see for example sequences that are illustrative members of the family (SEQ ID NO: 1-3)). In some embodiments, for example, where AGOG is the ssDNA cleaving enzyme, the modified nucleotide may be consumed in the cleavage reaction such that neither of the ssDNA fragments generated will comprise the modified nucleotide present in the substrate ssDNA.

These enzymes may be reagents that are lyophilized, purified, and/or immobilized. For ease of purification or handling, these enzymes may be fused to affinity binding proteins. The reagent enzymes may be in a storage buffer or before during or after addition to the ss oligonucleotide, in a reaction buffer.

Examples of modified nucleotides include deoxyuridine, deoxyinosine, 8-oxoguanine, apurinic site, tetrahydrofuran site, NMP, apyridimic NMP, rNMP and deoxyxanthosine, or thymine glycol. Other examples may include benzyl guanine and modifications thereof where the modification may include a label for detection or mobilization.

Examples of solid substrates for attaching ssDNA include for example, bead, arrays, plates or papers, microfluidic devices, tubes, and/or columns.

Molecular biology uses for ssDNA is continually increasing in ways that may utilize a dsDNA complement. For example, ssDNA can be used to hybridize to a nucleic acid (RNA, dsDNA, cDNA); immobilized ssDNA can be hybridized to target nucleic acids and extended to couple the sequence to a solid support rather than relying on hybridization alone for capture. SsDNA may also be used for synthesis and other applications where a single stranded complement is not required.

Examples use oligonucleotide synthesis, gene assembly and nucleic acid capture and enrichment, Next Generation Sequencing (NGS) or Sanger sequencing or by other methods such as quantitative polymerase chain reaction (qPCR) or dideoxy PCR (ddPCR). Cleaved oligos can be used for gene assembly methods (Klein, et al., Nucleic Acids Res, 44, e43 (2016)), PCR primers or other techniques.

Kits may be provided for use in the various contexts described above. For example, a kit to capture polyA mRNA on beads for reverse transcription or for nucleic acid capture and release as part or all of a sequencing workflow may include a ssDNA cleaving endonuclease (EndoQ for dU or dI, AGOG for 8-oxoG) and one or more of the following components: streptavidin beads, a capture oligonucleotide [biotin-primer(dU or dI or 8oxoG or dX)-poly(T)], reverse transcriptase, dNTPs; NEBNext® Ultra II Library Preparation Kit (New England Biolabs, Ipswich, Mass.).

The reagents in the kits may be stored as separate components in different tubes or may form a mixture as most convenient for the user and the use. Instructions are also included in the kit.

All publications, patents, and patent applications mentioned in this specification are herein incorporated by reference to the same extent as if each individual publication, patent, or patent application was specifically and individually indicated to be incorporated by reference.

### EXAMPLES

### Example 1: 9° N EndoQ Has ssc≥dsDNA-dU Cleavage Activity

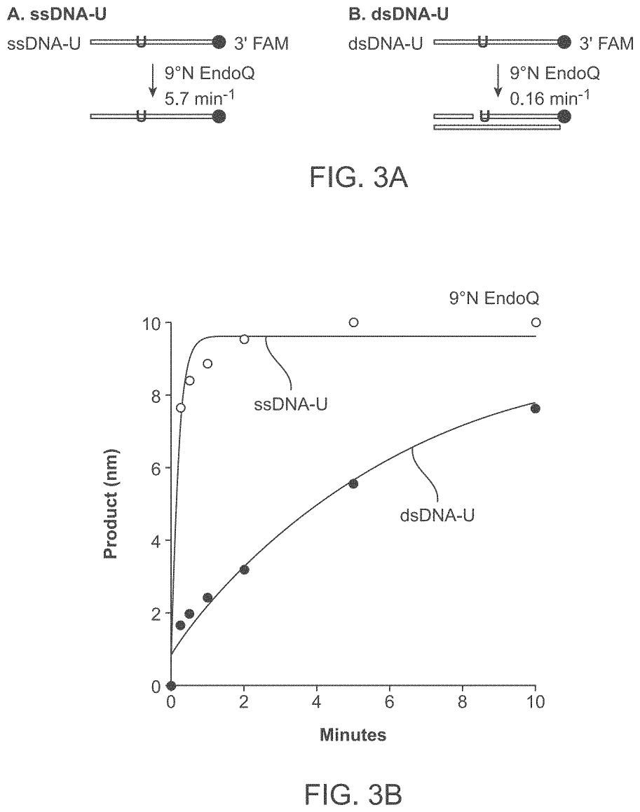

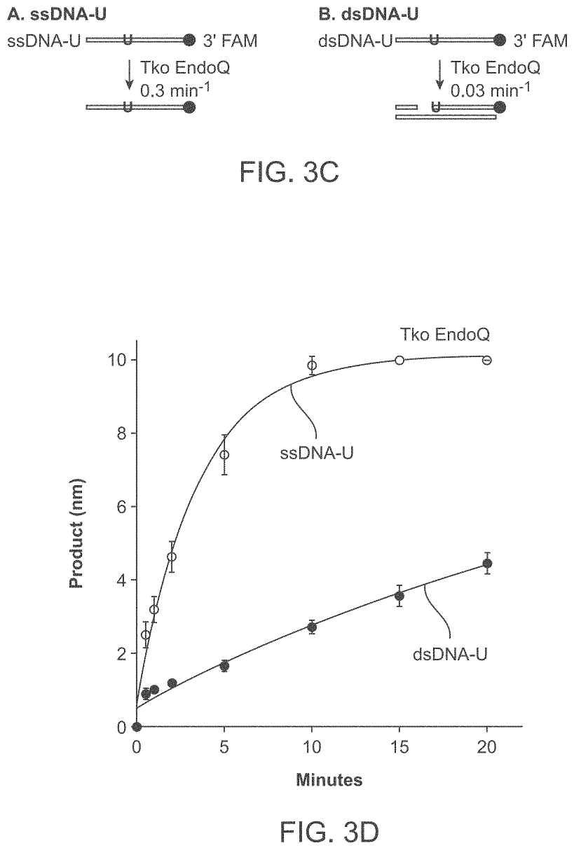

The efficiency of 9° N EndoQ cleavage of uracil was determined in ssDNA or dsDNA templates (schematically depicted in FIG. 3A). A FAM-labeled ssDNA substrate (10 nM) containing a dU (ssDNA-dU: Biotin-TGGAGATTTTGATCACGGTAACCdUATCAGAATGACAACAAGCCCGAATTCACCCAGGAGG-FAM) in 1× CutSmart® buffer (New England Biolabs, Ipswich, Mass.) (50 mM Potassium Acetate, 20 mM Tris-acetate, pH 7.9@25° C., 10 mM Magnesium Acetate, 100 μg/ml BSA) was incubated with 9° N EndoQ (1 nM final concentration) at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 3B). The rate of ssDNA-dU cleavage (m3) was 5.7 min⁻¹ (Table 1).

Similarly, the rate of dsDNA-dU cleavage by 9° N EndoQ was determined. Substrate dsDNA-dU was prepared by annealing a FAM-labeled ssDNA substrate (10 nM) containing a dU (ssDNA-dU: Biotin-TGGAGATTTTGATCACGGTAACCdUATCAGAATGACAACAAGCCCGAATTCACCCAGGAGG-FAM) to an unlabeled complementary template oligonucleotide (12 nM) (CCTCCTGGGTGAATTCGGGCTTGTTGTCATTCTGATAGGTTACCGTGATCAAAATCTCCA) in 1× CutSmart buffer using standard annealing protocols. Cleavage reactions (100 μl) were 10 nM dsDNA-U in 1× CutSmart buffer with 10 nM 9° N EndoQ at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 3B). The rate of dsDNA-dU cleavage (m3) was 0.16 min⁻¹ (Table 1).

### Example 2: Tko EndoQ Has ss≥dsDNA-dU Cleavage Activity

The efficiency of Tko EndoQ cleavage of dU was determined in ssDNA or dsDNA templates (schematically depicted in FIG. 3C). A FAM-labeled ssDNA substrate (10 nM) containing a dU (ssDNA-dU: Biotin-TGGAGATTTTGATCACGGTAACCdUATCAGAATGACAACAAGCCCGAATTCACCCAGGAGG-FAM) in 1× CutSmart buffer (50 mM Potassium Acetate, 20 mM Tris-acetate, pH 7.9@25° C., 10 mM Magnesium Acetate, 100 μg/ml BSA) was incubated with Tko EndoQ (1 nM final concentration) at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 3C). The rate of ssDNA-dU cleavage (m3) was 0.3 min⁻¹ (Table 1).

Similarly, the rate of dsDNA-dU cleavage by Tko EndoQ was determined. Substrate dsDNA-dU was prepared by annealing a FAM-labeled ssDNA substrate (10 nM) containing a dU (ssDNA-dU: Biotin- TGGAGATTTTGATCACGGTAACCdUATCAGAATGACAACAAGCCCGAATTCACCCAGGAGG-FAM) to an unlabeled complementary template oligonucleotide (12 nM) (CCTCCTGGGTGAATTCGGGCTTGTTGTCATTCTGATAGGTTACCGTGATCAAAATCTCCA) in 1× CutSmart buffer using standard annealing protocols. Cleavage reactions (100 μl) were 10 nM dsDNA-U in 1× CutSmart buffer with 10 nM Tko EndoQ at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 3D). The rate of dsDNA-dU cleavage (m3) was 0.03 min⁻¹ (Table 1)(see FIG. 3D).

### Example 3: 9° N EndoQ Has ss≥dsDNA-dI Activity

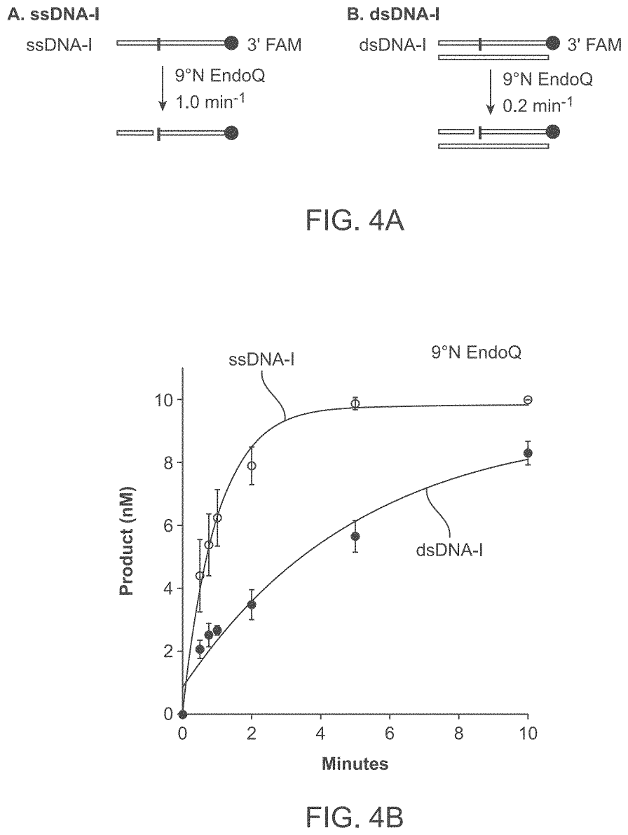

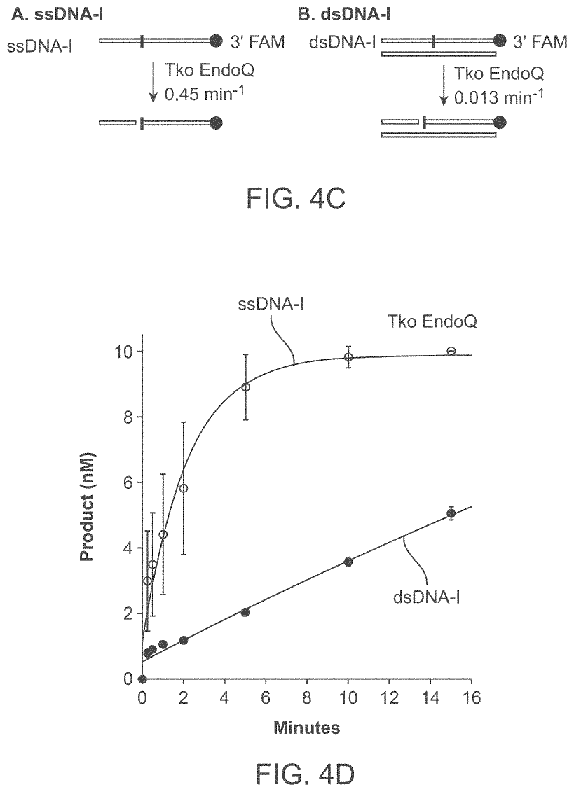

The efficiency of 9° N EndoQ cleavage of inosine was determined in ssDNA or dsDNA templates (schematically depicted in FIG. 4A). A FAM-labeled ssDNA substrate (10 nM) containing an dI (ssDNA-dI: Biotin-TGGAGATTTTGATCACGGTAACCdIATCAGAATGACAACAAGCCCGAATTCACCCAGGAGG-FAM) in 1× CutSmart buffer (50 mM Potassium Acetate, 20 mM Tris-acetate, pH 7.9@25° C., 10 mM Magnesium Acetate, 100 μg/ml BSA) was incubated with 9° N EndoQ (1 nM final concentration) at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 4B). The rate of ssDNA-dI cleavage (m3) was 1.0 min⁻¹ (Table 1).

Similarly, the rate of dsDNA-dI cleavage by 9° N EndoQ was determined. Substrate dsDNA-dI was prepared by annealing a FAM-labeled ssDNA substrate (10 nM) containing a dI(ssDNA-dI: Biotin-TGGAGATTTTGATCACGGTAACCdIATCAGAATGACAACAAGCCCGAATTCACCCAGGAGG-FAM) to an unlabeled complementary template oligonucleotide (12 nM) (CCTCCTGGGTGAATTCGGGCTTGTTGTCATTCTGATTGGTTACCGTGATCAAAATCTCCA) in 1× CutSmart buffer using standard annealing protocols. Cleavage reactions (100 μl) were 10 nM dsDNA-dI in 1× CutSmart buffer with 10 nM 9° N EndoQ at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 4B). The rate of dsDNAd-l cleavage (m3) was 0.2 min⁻¹ (Table 1) (see FIG. 4B).

### Example 4: Tko EndoQ Has ss≥dsDNA-dI Activity

The efficiency of Tko EndoQ cleavage of inosine was determined in ssDNA or dsDNA templates (Schematically depicted in FIG. 4C). A FAM-labeled ssDNA substrate (10 nM) containing an idI (ssDNA-dI: Biotin-TGGAGATTTTGATCACGGTAACCdIATCAGAATGACAACAAGCCCGAATTCACCCAGGAGG-FAM) in 1× CutSmart buffer (50 mM Potassium Acetate, 20 mM Tris-acetate, pH 7.9@25° C., 10 mM Magnesium Acetate, 100 μg/ml BSA) was incubated with Tko EndoQ (1 nM final concentration) at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 4D). The rate of ssDNA-dI cleavage (m3) was 0.45 min⁻¹ (Table 1).

Similarly, the rate of dsDNA-dI cleavage by Tko EndoQ was determined. Substrate dsDNA-dI was prepared by annealing a FAM-labeled ssDNA substrate (10 nM) containing a dI(ssDNA-dI: Biotin-TGGAGATTTTGATCACGGTAACCdIATCAGAATGACAACAAGCCCGAATTCACCCAGGAGG-FAM) to an unlabeled complementary template oligonucleotide (12 nM) (CCTCCTGGGTGAATTCGGGCTTGTTGTCATTCTGATTGGTTACCGTGATCAAAATCTCCA) in 1× CutSmart buffer using standard annealing protocols. Cleavage reactions (100 μl) were 10 nM dsDNA-dI in 1× CutSmart buffer with 10 nM Tko EndoQ at 65° C. Reaction aliquots (10 μl) were removed at 0, 0.25, 0.5, 1, 2, 5, 10 and 20 minutes and mixed with 10 μl 50 mM EDTA to halt the reaction. Reaction products were separated and analyzed by capillary electrophoresis. The fraction of cleaved product was calculated, plotted and fit to an exponential rise equation (y=m1+m2*(1−exp(−m3*x)) (FIG. 4D). The rate of dsDNA-dI cleavage (m3) was 0.013 min⁻¹ (Table 1) (see FIG. 4D).

### Example 5: AGOG Has ss≥dsDNA-8oxoG Activity

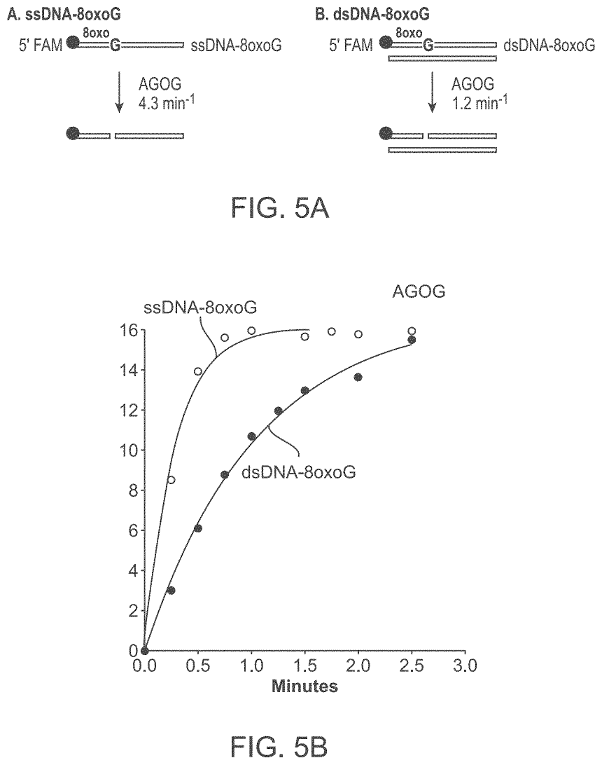

The efficiency of AGOG cleavage of 8-oxoG was determined in ssDNA or dsDNA templates (Schematically depicted in FIG. 5A). The ssDNA-8oxoG substrate was (FAM-TGGAGATTTTGATCACGGTAACC(8oxoG)ATCAGAATGACAACAAGCCCGAATTCACCCAGGAGG-ROX). The dsDNA-8oxoG containing substrates were prepared by annealing 1 uM of the 60-nt labeled-lesion containing oligonucleotide (FAM-TGGAGATTTTGATCACGGTAACC(8oxoG)ATCAGAATGACAACAAGCCCGAATTCACCCAGGAGG-ROX) to 1.25 uM of the 60-nt complementary oligonucleotide (CCTCCTGGGTGAATTCGGGCTTGTTGTCATTCTGATCGGTTACCGTGATCAAAATCTCCA) in 1× annealing buffer (10 mM Tris-HCl pH 7.5 and 100 mM NaCl) at 85° C. for 5 minutes and allowing to slowly cool to room temperature.

To determine the rates of glycosylase and lyase activity of AGOG on ssDNA-8oxoG or dsDNA-8oxoG, single-turnover kinetic assays were performed with AGOG in excess of the substrate. For each timepoint, a 10 μL reaction was made in 1× ThermoPol® buffer (New England Biolabs, Ipswich, Mass.) containing 20 nM of substrate ssDNA-8oxoG or dsDNA-8oxoG. To start the reaction, 100 nM AGOG (final concentration) was added. A control experiment demonstrated that the substrate was saturated with a 5-fold excess of AGOG. When measuring the base removal step of the reaction, the reactions were stopped at the appropriate time points with equal volume 0.1 N NaOH, 0.25% SDS and then neutralized with equal volume 1 M Tris-HCl pH 7.5. For measuring the rate of the total reaction, the reactions were stopped with equal volume 80% formamide, 50 mM EDTA. In all cases, the reactions were cleaned-up and analyzed using capillary electrophoresis as described above. The concentration of product was graphed as a function of time and fit to a single-exponential equation ((y=m1+m2*(1−exp(−m3*x))) to obtain the observed rate of substrate cleavage (k_{obs}) using KaleidaGraph (Synergy Software, Reading, Penn.). The rate of AGOG cleavage of ssDNA-8oxoG was 4.3 min⁻¹ and ssDNA-8oxoG was 1.2 min⁻¹ (see FIG. 5B).

### Example 6: 9° N RNaseH2 Has ss<dsDNA Activity

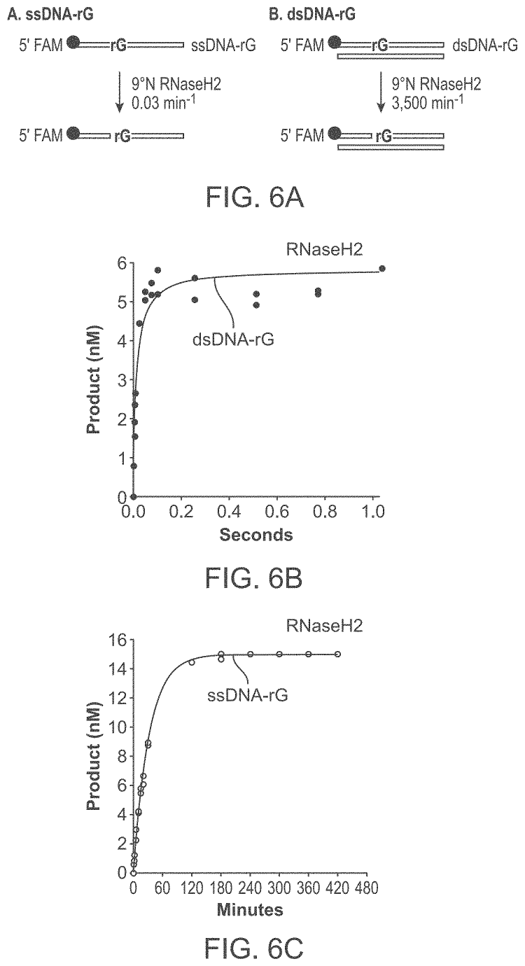

The efficiency of 9° N RNaseH2 cleavage of rG was determined in ssDNA or dsDNA templates (schematically depicted in FIG. 6A) as described in Heider, et al., J Biol Chem, 292, 8835-8845 (2017). Reaction products were separated and analyzed by capillary electrophoresis. The fraction of cleaved product was calculated, plotted and fit to an exponential rise equation (y=m1+m2*(1−exp(−m3*x)) (FIGS. 6B and 6C). The rate of ssDNA-rG cleavage (m3) was 0.03 min⁻¹ (Table 1).

Similarly, the rate of dsDNA-rG cleavage by 9° N RNaseH2 was determined (Heider, et al., J Biol Chem, 292, 8835-8845 (2017)). The rate of dsDNA-l cleavage (m3) was 3,500 min-1 (Table 1 and FIGS. 6B and 6C). The ratio of ssDNA-rG:dsDNA-rG activity by 9° N RNaseH2 was 8.5×10−6 and thus 9° N RNaseH2 has ss<dsDNA-rG activity (see FIGS. 6B and 6C).

TABLE 1
Summary of the activity ratio of various thermophilic endonucleases
on modified ssDNA and dsDNA substrates.

| | | ssDNA | dsDNA | ssDNA/dsDNA |
|---|---|---|---|---|
| Enzyme | Substrate | (min⁻¹) | (min⁻¹) | activity ratio |
| Tko EndoQ | dU | 0.3 | 0.03 | 10 |
| 9°N EndoQ | dU | 5.7 | 0.16 | 35 |
| Tko EndoQ | dI | 0.45 | 0.013 | 35 |
| 9°N EndoQ | dI | 1.0 | 0.2 | 5 |
| AGOG | 8-oxo-dG | 4.3 | 1.2 | 3.5 |
| 9°N RNaseH2 | rN | 0.03 | 3,500 | 8.5 × 10⁻⁶ |

### Example 7: Cleavage of ssDNA-dU-beads with EndoQ

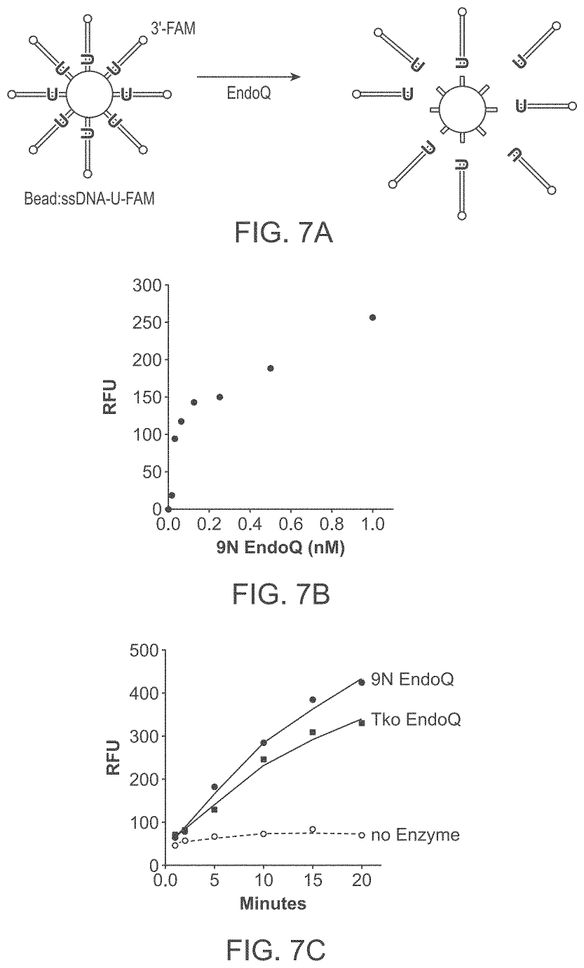

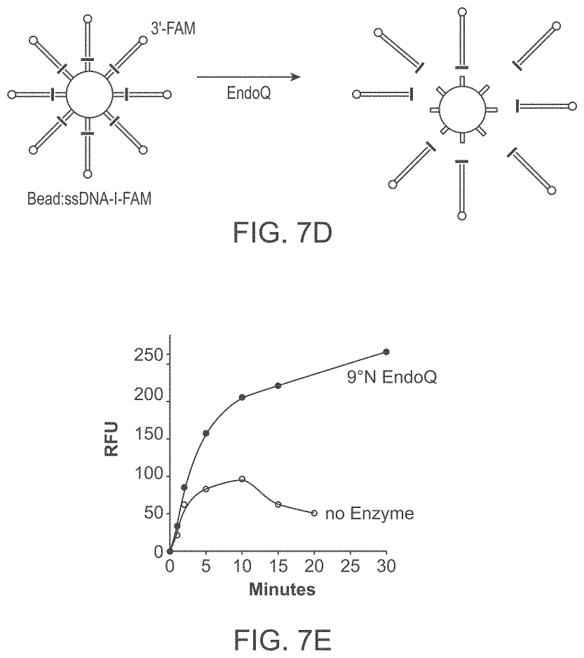

Biotin-ssDNA-dU-3′-FAM (1 μM) was attached to streptavidin magnetic beads. After washing unbound ssDNA with a wash buffer (0.5 NaCl, 20 mM TrisHCl, pH 7.5), 10 nM 9° N EndoQ or Tko EndoQ was added in 100 μl 1× CutSmart buffer and cleaved at dU to release the FAM-labeled product from the magnetic bead (Schematically depicted in FIG. 7A). The released FAM fluorescence was measured by a Molecular Devices plate reader (Molecular Devices, San Jose, Calif.) (FIG. 7B). FIGS. 7A-7E quantitates the cleaved ssDNA-dU-3′-FAM by (FIG. 7A) a titration of 9° N EndoQ or (FIG. 7B) by 9° N or Tko EndoQ over time. No Enzyme was added as a negative control (see FIG. 7A-C).

### Example 8: Cleavage of ssDNA-dI-beads with 9° N EndoQ

Biotin-ssDNA-dI-3′-FAM (1 μM) was attached to streptavidin magnetic beads. After washing unbound ssDNA with a wash buffer (0.5 NaCl, 20 mM TrisHCl, pH 7.5), 10 nM 9° N EndoQ was added in 100 μl 1× CutSmart buffer and cleaved at uracil to release the FAM-labeled product from the magnetic bead (Schematically depicted in FIG. 7D). The released FAM fluorescence was measured by a Molecular Devices plate reader. FIG. 7D-7E quantitate the cleaved ssDNA-dI-3′-FAM by 9° N EndoQ or a no enzyme control over time.

### Example 9: Cleavage of ssDNA-dU-beads with 9° N EndoQ

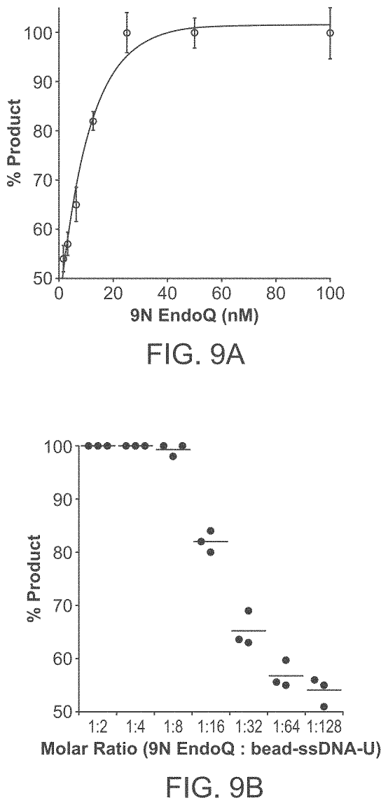

Biotin-ssDNA-dU-3′-FAM (1 μM) was attached to streptavidin magnetic beads and washed (5 times) to remove unbound ssDNA with a wash buffer (0.5 NaCl, 20 mM TrisHCl, pH 7.5). A 50 μl reaction with 200 nM ssDNA-dU-beads, 1× CutSmart buffer and various amounts (100 nM to 3.16 nM) of 9° N EndoQ was incubated at 65° C. for 20 minutes. The ratio of EndoQ to ssDNA-dU was 1:1, 1:2, 1:4, 1:8, 1:16, 1:32, 1:64 and 1:128. EndoQ cleaved at uracil to release the FAM-labeled product from the magnetic bead (schematically depicted in FIG. 7A). The total and released FAM fluorescence was measured by a Molecular Devices plate reader. The % Product was calculated by the equation: % P=100*(released FAM-ssDNA-dU/FAM-ssDNA-dU+FAM-ssDNA-dU-bead). FIG. 7B-7C quantitates the cleaved ssDNA-dU-3′-FAM by 9° N EndoQ. At least 90% of the immobilized ssDNA-U was cleaved using a less than or equal 1:1 ratio of EndoQ:immobilized ssDNA-dU (see FIG. 9A-9B).

### Example 10: Cleavage of ssDNA-dU-plate with EndoQ

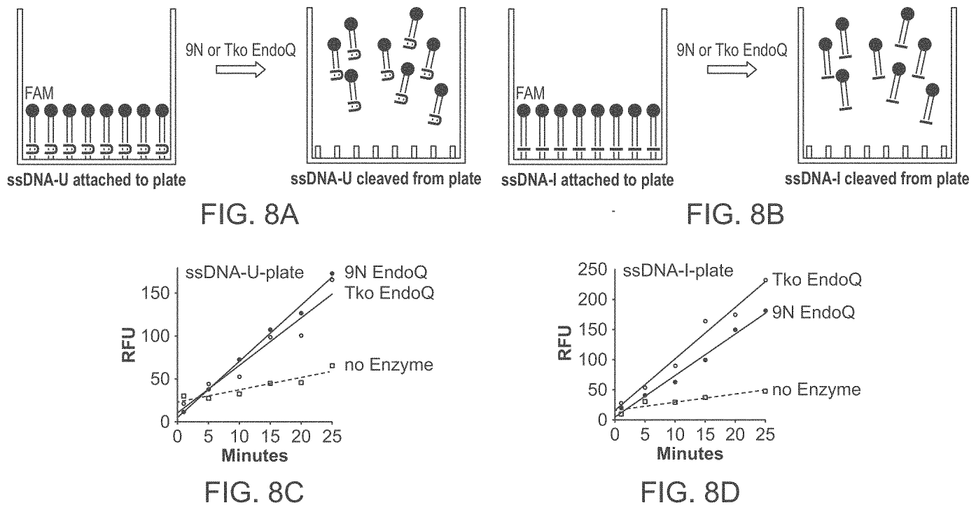

Biotin-ssDNA-dU-3′-FAM was attached to a streptavidin coated polystyrene plate (Thermo Nunc Immobilizer Streptavidin C8) by incubating 0.5 μM Biotin-ssDNA-dU-3′-FAM in 100 μl wash buffer (0.5 NaCl, 20 mM TrisHCl, pH 7.5) for 30 minutes at 25° C. Unbound ssDNA was washed off with a wash buffer (0.5 NaCl, 20 mM TrisHCl, pH 7.5). 9° N EndoQ or Tko EndoQ (10 nM) was added in 1× CutSmart Buffer to cleaved at dU to release the FAM-labeled product from the plate (schematically depicted in FIG. 8A-8D). The released FAM fluorescence was measured by a Molecular Devices plate reader (FIG. 8B).

### Example 11: Cleavage of ssDNA-dI-plate with EndoQ

Biotin-ssDNA-dI-3′-FAM was attached to a streptavidin coated polystyrene plate (Thermo Nunc Immobilizer Streptavidin C8) by incubating 0.5 μM Biotin-ssDNA-dI-3′-FAM in 100 μl wash buffer (0.5 NaCl, 20 mM TrisHCl, pH 7.5) for 30 minutes at 25° C. Unbound ssDNA was washed off with a wash buffer (0.5 NaCl, 20 mM TrisHCl, pH 7.5). 9° N EndoQ or Tko EndoQ (10 nM) was added in 1× CutSmart Buffer to cleaved at dI to release the FAM-labeled product from the plate (Schematically depicted in FIG. 8C). The released FAM fluorescence was measured by a Molecular Devices plate reader and the results are shown in FIG. 8D.

Tko EndoQ:
(SEQ ID NO: 1)
MIVDADLHIHSRYSKAVSKAMTIPNLAENARFKGL
EMVGTGDILNPNWEKELLKYTKKVDEGTYERNGIR
FLLTTEVEDTRRVHHVLIFPNIETVREMRERLKPY
SSDIESEGRPHLTLSAAEIADIANELDVLIGPAHA
FTPWTSLYKEYDSLKEAYNGAKIHFLELGLSADSE
MADMIKAHHKLTYLSNSDAHSPMPHRLGREFNRFE
VNEATFEEIRKAILKRGRKIVLNAGLDPRLGKYHL
TACSRCYTKYSLEEAKAFRWKCPKCGGRIKKGVRD
RILELADTTERPKDRPPYLHLAPLAEIIAMVLGKG
VETKAVRLVWERFLREFGSEIRVLVDVPVEELAKV
HEEVAKAVWAYRKGKLIVISGGGGKYGEIKLPDEV
RNARIEDLETIEVEVPNVEEKPKQRSITEFLRKSN
K
9°N EndoQ
(SEQ ID NO: 2)
MLVDADLHLHSRYSKAVSKAMTIPNLAQNARFKGL
GLVGTGDILNPHWEAELLRYAKKVDEGTYELNGIR
FLLTTEVEDNRRVHHVLIFPSIETVREMREILKRY
STDIETEGRPHLSLSAAEIADIANDLDILIGPAHA
FTPWTSLYKEYDSLKEAYRNARVHFLELGLSADSE
MADMIKAHHRLTYLSNSDAHSPMPHRLGREFNRFE
VEEVTFEEVRKAILRRGGRRIVLNAGLDPRLGKYH
LTACSRCYAHYSLGEAKAFKWKCPKCGGRIKKGVK
DRILELADTEERPKDRPPYLRLAPLAEIISMVIGK
GIETKAVRLIWERFLRDFGSEIRVLVDVPVKELAN
VHEEVAKAIWAYRNGKLIVIPGGGGKYGEIKLPEE
IRKARVEDLESVEVEIPEETEKPRQRSITDFLK
Tk AGOG:
(SEQ ID NO: 3)
MSLERFVKIKYQTNEEKADKLVEGLKELGIECARI
IEEKVDLQFDALRHLRENLNDDETFIKLVIANSIV
SYQLSGKGEDWWWEFSKYFSQNPPEKSIVEACSKF
LPSSRTNRRLVAGKIKRLEKLEPFLNSLTLQELRR
YYFENMMGLRNDIAEALGSPKTAKTVVFAVKMFGY
AGRIAFGEFVPYPMEIDIPEDVRIKAYTERITNEP
PVSFWRRVAEETGIPPLHIDSILWPVLGGKREVME
RLKKVCEKWELVLELGSL

What is claimed is:

1. A method comprising:
(a) combining a single stranded DNA (ssDNA) comprising a modified nucleotide with a single stranded DNA cleavage enzyme capable of cleaving the ssDNA at the modified nucleotide in the ssDNA to generate a first ssDNA fragment having a 3′OH and a second ssDNA fragment having the modified nucleotide wherein the ratio of enzyme to DNA substrate is less than 1:1 molar ratio and
(b) cleaving at least 95% of the ssDNA at the modified nucleotide.

2. A method, comprising:
(a) combining (i) a single stranded DNA (ssDNA) (1) immobilized on a substrate and (2) comprising a modified nucleotide with (ii) a ssDNA cleavage enzyme capable of cleaving the DNA at the modified nucleotide in the ssDNA to generate after cleavage, a first ssDNA fragment having a 3′OH and a second ssDNA fragment having the modified nucleotide; and
(b) cleaving the immobilized ssDNA to release the second ssDNA fragment from the substrate.

3. A method according to claim 1, wherein (b) further comprises cleaving at least 95% of the ssDNA in less than 60 minutes.

4. The method according to claim 1, wherein the ssDNA comprising a modified nucleotide further comprises the modified nucleotide proximate to the 5′ end of the ssDNA.

5. The method according claim 1, wherein the ssDNA is immobilized on a solid support.

6. The method according to claim 1, wherein cleaving further comprises cleaving the immobilized DNA proximate to the modified nucleotide with the ssDNA cleavage enzyme and releasing from the substrate a fragment of the ssDNA comprising the modified nucleotide and nucleotides 3′ to the modified nucleotide.

7. The method according to claim 1 further comprising, prior to step (a) generating the ssDNA by reverse transcribing an RNA.

8. The method according to claim 1, wherein the ssDNA containing a modified nucleotide proximate to its 5′ end further comprises a label at a 3′ end.

9. The method according to claim 8, wherein the label is a fluorescent tag.

10. The method according to claim 5, wherein the solid support is a bead.

11. The method according to claim 5, wherein the solid support is plastic plate with wells.

12. The method according to claim 5, wherein the solid support is a two-dimensional surface on which the ssDNA forms an array.

13. The method according to claim 1, wherein the sssDNA cleavage enzyme comprises a thermophilic endonuclease.

14. The method according to claim 13, wherein the thermophilic endonuclease is an archaeal endonuclease.

15. The method according to claim 14, wherein the thermophilic endonuclease is an EndoQ.

16. The method according to claim 14, wherein the ssDNA cleavage enzyme is AGOG.

17. The method according to claim 1, wherein the ssDNA cleavage enzyme comprises a fusion protein.

18. The method according to claim 16, wherein the ssDNA cleavage enzyme further comprises a SNAP-tag.

19. The method according to claim 18, wherein the SNAP-tag is bound to a solid substrate.

20. The method according to claim 1, wherein the modified nucleotide is an 8-oxoG.

21. The method according to claim 1, wherein the modified nucleotide is deoxyuridine.

22. The method according to claim 1, wherein the modified nucleotide is deoxyinosine.

23. The method according to claim 1, wherein the single stranded oligonucleotide is a product of ssDNA synthesis and optionally contains a barcode of randomly generated nucleotides.

24. The method according to claim 1, wherein the ssDNA is an aptamer.

25. The method according to claim 1, wherein the ssDNA synthesis is chemical or enzymatic.

26. A composition comprising an artificial mixture of a ssDNA-cleaving archaeal endonuclease or glycosylase and a synthetic DNA substrate comprising a modified nucleotide.

27. The composition according to claim 26, wherein the synthetic DNA substrate is immobilized on a solid substrate.

28. The composition according to claim 27, where the solid substrate is selected from a bead, a well in a multi-well dish and a two-dimensional array surface.

29. The composition according to claim 26, wherein the modified nucleotide is selected from the group consisting of deoxyuridine, deoxyinosine, 8-oxoG, deoxyxanthosine and tetrahydrofuran site.

30. (canceled)

31. The composition according to claim 26, wherein the fusion protein comprises a SNAP-tag.
